(19) United States
(12) Patent Application Publication
(10) Pub. No.: US 2018/0029015 A1
(43) Pub. Date: Feb. 1, 2018

(54) CATALYTIC COMPOSITE AND IMPROVED PROCESS FOR DEHYDROGENATION OF HYDROCARBONS

(71) Applicant: SABIC GLOBAL TECHNOLOGIES B.V., BERGEN OP ZOOM (NL)

(72) Inventors: KARTICK CHANDRA MONDAL, Bangalore (IN); DIPABALI ROY CHOWDHURY, Bangalore (IN); VELAYUTHAM SARAVANAN, Bangalore (IN)

(21) Appl. No.: 15/552,736

(22) PCT Filed: Feb. 22, 2016
(86) PCT No.: PCT/IB2016/050947

§ 371 Date: Aug. 22, 2017

Related U.S. Application Data
(60) Provisional application No. 62/119,576, filed on Feb. 23, 2015.

(51) Int. Cl. B01J 23/652; C07C 5/32; B01J 21/04; B01J 8/02; B01J 23/18; B01J 35/02

(52) U.S. Cl. CPC B01J 23/6522; B01J 23/18; B01J 35/026; B01J 21/04; B01J 8/02; C07C 5/325; B01J 2208/027; C07C 2523/652; C07C 2523/644

(57) ABSTRACT

A catalytic composite for a cyclic process of adiabatic, non-oxidative dehydrogenation of an alkane into an olefin, comprising a dehydrogenation catalyst, a semimetal and a carrier supporting the catalyst and the semimetal. During the reduction and/or regeneration stages of the adiabatic process, the semimetal releases heat which can be used to initiate the dehydrogenation reactions, which are endothermic in nature, thereby reducing the need for hot air flow and combustion of coke as heat input. The semi-metal is inert towards the dehydrogenation reaction itself, alkane feed and olefin product as well as other side reactions of the cyclic process such as cracking and decoking.

### BACKGROUND OF THE INVENTION

### Technical Field

The present disclosure relates to catalytic composites for dehydrogenation processes. More specifically, the present disclosure relates to catalytic composites incorporating at least one catalytically inactive semimetal and adiabatic, non-oxidative, cyclic dehydrogenation processes wherein these catalytic composites are used.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The dehydrogenation of hydrocarbons, involves the breaking of two carbon-hydrogen (C—H) bonds with the simultaneous formation of a hydrogen molecule (H₂) and a molecule containing a double carbon-carbon bond (C═C). The double bond is a highly reactive point that permits the use of double bond-containing molecules as intermediates for the production of typical petrochemical products such as polymers. Dehydrogenation reactions that are of significant industrial interest include dehydrogenation of low paraffins (C₂-C₅ alkanes) to produce corresponding olefins or alkenes, dehydrogenation of C₁₀-C₁₅ linear paraffins to yield linear-alkyl-benzenes and ethyl benzenes that provide starting points for the production of polystyrene plastics.

Dehydrogenation of alkanes to olefins can generally be classified as either oxidative or non-oxidative reactions. Disadvantages associated with oxidative dehydrogenation include high exothermicity and low desired product selectivity and quality. Non-oxidative processes (i.e., direct dehydrogenation or catalytic dehydrogenation), on the other hand, are endothermic and can suffer from the requirement of a continuous heat supply to initiate the endothermic reaction. The temperatures that are required to shift the equilibria favorably to alkene products during direct dehydrogenation can promote rapid deactivation of the catalyst by coking, resulting in the need for frequent catalyst regeneration. These high temperatures can also lead to thermal cracking of the alkanes, which can lead to undesirable non-selective side reactions that result in formation of byproducts.

One such non-oxidative dehydrogenation process is the Catofin process. In the Catofin process, the dehydrogenation of the hydrocarbon feedstock (e.g. propane, n-butane, isobutane, and isopentane) and the regeneration of the catalyst or decoking, alternate in a cyclic or repetitive manner Both dehydrogenation and regeneration are designed to run adiabatically, with the catalyst on the hydrocarbon feed for very short cycles (7-15 minutes (min), preferably 2-25 min, 5-20 min, or 8-10 min), followed by regeneration of the catalyst for a similar period of time. Since the Catofin process is designed to be adiabatic, and in order to prevent a decrease in alkane conversion, the consumption of heat during the endothermic dehydrogenation reaction needs to be closely in balance with the heat restored to the bed during the exothermic regeneration cycles.

In traditional Catofin processes, the reactor or the catalyst bed is purged with hot air during regeneration cycle in order to reheat the catalyst and remove coke which has been deposited on the catalyst bed during the endothermic dehydrogenation step. However, since the duration of the regeneration cycle is short, there is a strong likelihood for the formation of a vertical temperature gradient and pressure drop across the catalyst bed, which adversely affects the overall yield of the olefin product. Hence, with hot air flow and combustion of coke as the sole heat sources, heat input to the catalyst bed remains a critical limiting factor to Catofin dehydrogenation processes.

More recently, an alternative approach towards heat transfer to the fixed Catofin catalyst bed was developed. Harnessing the fact that the Catofin process operates in a cyclic, reduction/oxidation mode, a material that is referred to as “heat generating material” (HGM) that functions as a catalyst additive material was developed. The heat generating material is mounted on a catalyst support and meets several key performance parameters such as (i) the ability to produce heat in situ while remaining inactive or inert to the hydrocarbon or alkane feed and the olefin products, and (ii) the absence of any negative impact on the activity, selectivity or lifetime of the catalyst (U.S. Pat. Nos. 7,622,623 and 7,973,207; Oviol, L, Bruns, M, Fridman, V, Merriam, J, Urbancic, M, “Mind the gap”, published by Clariant Catalysis and Energy, formerly Süd-Chemie). Nevertheless, the heat-generating material is not inert towards the reducing and/or oxidizing conditions of a Catofin process. The heat generating material is a metal selected from copper, chromium, molybdenum, vanadium, yttrium, scandium, tungsten, manganese, iron, cobalt, nickel, silver (transition metals, bismuth (post-transition metal) and cerium (lanthanide metal).

The use of some of the metals listed above, such as bismuth, molybdenum, vanadium, as reactive catalytic material or a promoter in dehydrogenation processes other than the Catofin process (i.e. non-cyclic, non-adiabatic and therefore different reaction conditions) has been described in U.S. Pate. Nos. 5,527,979 and 4,524,236 and European Patent EP 2143701 B1 (each incorporated herein by reference in its entirety).

It is desirable to provide novel materials that generate heat in a cost-effective manner for improved adiabatic, non-oxidative, cyclic dehydrogenation processes. These heat generating materials desirably remain inert towards the dehydrogenation reaction and any other side reactions (e.g., cracking or coking), the feed, and the product.

### BRIEF SUMMARY OF THE INVENTION

Disclosed herein are a catalyst composition and methods of using the same.

A catalytic composite suitable for a cyclic process of adiabatic, non-oxidative dehydrogenation of an alkane into an olefin is disclosed herein. The catalytic composite can comprise a dehydrogenation catalyst, a semimetal and a carrier supporting the dehydrogenation catalyst and the semimetal. The semimetal is inert towards the dehydrogenation, and releases heat in situ when exposed to at least one of a reducing stage and an oxidizing stage of the cyclic process.

A fixed bed catalyst can be packed with at least one layer comprising the catalytic composite.

An adiabatic, fixed-bed reactor can comprise a fixed bed catalyst packed with at least one layer comprising the catalytic composite.

A process of producing an olefin by adiabatic, non-oxidative dehydrogenation of an alkane can comprise: (a) preparing a fixed bed catalyst comprising at least one layer of a catalytic composite, the catalytic composite comprising a dehydrogenation catalyst, a semimetal and a carrier supporting the dehydrogenation catalyst and the semimetal; (b) reducing the fixed bed catalyst to generate a first heat supply, which is released by the semimetal, that is passed into the fixed catalyst bed; (c) contacting a feed stream comprising the alkane with the fixed bed catalyst to endothermically dehydrogenate the alkane, wherein the thermal energy consumed by the dehydrogenation is at least partially provided by the first heat supply; (d) steam purging and oxidizing the fixed bed catalyst to regenerate the fixed catalyst bed and oxidize the semimetal and to optionally generate a second heat supply; and (e) optionally repeating (b) to (d) for multiple cycles.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an XRD spectrum of calcined 12 wt. % Sb₂O₃/γ-Al₂0₃.

FIG. 2 is a flowchart illustrating the process of preparing a catalytic composite with a semimetal and commercial Catofin STD catalyst according to one embodiment.

FIG. 3 is schematic representation of a cross-sectional view of a fixed catalyst bed according to one embodiment, showing an arrangement of the dehydrogenation catalyst, carrier and heat-generating semimetal.

FIG. 4A is a schematic diagram of a fixed catalyst bed with multiple layers according to one embodiment.

FIG. 4B is a schematic diagram of a fixed catalyst bed with multiple layers according to another embodiment.

FIG. 4C is a schematic diagram of a fixed catalyst bed where heat-generating semimetal particles are thoroughly mixed with carrier-supported dehydrogenation catalyst particles.

### DETAILED DESCRIPTION OF THE EMBODIMENTS

Referring now to the drawings, wherein like reference numerals designate identical or corresponding parts throughout the several views.

In the present disclosure, a novel catalytic composite intended for use in non-oxidative aliphatic hydrocarbon dehydrogenation processes is provided. These dehydrogenation processes generally follow the typical Houdry Catofin process as described in U.S. Pat. No. 2,419,997, wherein light aliphatic hydrocarbons from crude oil, such as alkanes, are catalytically dehydrogenated to their corresponding mono- or di-olefins by a dehydrogenation catalyst, in an adiabatic, non-oxidative and repetitive manner. Advantageously, these dehydrogenation processes take place in equipment or a reactor that contains at least one fixed catalyst bed. The fixed catalyst bed is packed with the catalytic composite of the present invention. The catalytic composite, which is in pellet form in at least one embodiment, is held in place in the packed bed and does not move with respect to a fixed reference frame. In certain embodiments, the fixed catalyst bed can include multiple layers of catalytic composites of different compositions or different dehydrogenation catalysts. The Catofin process can operate at atmospheric pressure or under partial or slight vacuum at 0.35-0.7 bar pressure.

Examples of aliphatic hydrocarbons that the catalytic composite of the present invention can react upon include but are not limited to C₂-C₂₀, preferably, C₂-0₅ alkanes. For example, propane, n-butane, isobutene and isopentane can be catalytically dehydrogenated into propylene, butadiene, isobutylene and isoprene respectively. These olefin products provide starting points for production of other useful thermoplastic polymers and compounds. For example, butadiene and isoprene are starting materials for the production of synthetic rubber. The octane booster compound, methyl-tert-butyl ether (MTBE), which is added to gasoline to increase the octane rating of the gasoline, can be produced from isobutylene.

As used herein, a “catalytic composite”, which is also called “catalyst composite”, “catalytic/catalyst composition”, “heteregeneous catalyst” or “composite catalyst”, refers to a composite material that is made up of two or more constituent materials, elements, parts or components with significantly different physical or chemical properities, that when combined, produce a composite material that possesses, among other properties and characteristics, the ability to increase or accelerate the rate of a dehydrogenation reaction, for example, in a Catofin process. In some embodiments, the catalytic composite according to the present disclosure further includes the ability to generate or release heat in situ in at least one stage of a Catofin process. The heat produced can be used to initiate and/or propagate the endothermic dehydrogenation reaction.

The catalytic composite of the present disclosure includes at least three main components: a dehydrogenation catalyst, a heat-generating semimetal and a solid, inert material that serves as a carrier supporting the catalyst and the semimetal. The dehydrogenation catalyst is selected from a noble metal or a Group VII metal such as platinum that is optionally alloyed with tin (e.g. PtSn, PtSn₂, Pt₂Sn₃ amd Pt₃Sn), a transition metal such as chromium, iron and copper, an oxide and a mixture and/or an alloy thereof, and a post-transition metal such as gallium, an oxide and/or an alloy thereof. In certain embodiments, the dehydrogenation catalyst is chromium-based (i.e. chromium/chromium oxide or chromia). The dehydrogenation catalyst must be able to accept repeated cycles of the Catofin process which alternate between reducing and oxidizing atmospheres. In certain embodiments, the dehydrogenation catalyst can be regenerated using steam. In some embodiments, the average particle size of the dehydrogenation catalyst exceeds 100 nm, for example, 0.1-100 μm, preferably 10-90 μm, more preferably 25-75 μm. In other embodiments, the dehydrogenation catalyst can be classified as a nanocatalyst or a nanomaterial-based catalyst wherein at least one dimension of the catalyst particle is of nanoscale and the average particle size is 1-100 nm, preferably 10-90 nm, more preferably 20-75 nm. Particle sizes can be determined by conventional techniques known in the art, such as scanning electron microscopy (SEM), transmission electron microscopy (TEM), X-ray diffraction analysis (XRD), and combinations comprising at least one of the foregoing.

The dehydrogenation catalyst is advantageously affixed to and distributed over a solid, inert material that is commonly referred to as a catalyst support or a carrier in the art, for the purpose of increasing or maximizing the surface area of the dehydrogenation catalyst. The carrier should also be thermally stable, being able to withstand high temperatures of up to 800° C., for example, 500-800° C., preferably 550-750° C., more preferably 600-700° C. A suitable catalyst support for the dehydrogenation catalyst is alumina-based, magnesia-based, silica-based, zirconia-based, zeolite-based or a combination thereof. In certain embodiments, an alumina-based catalyst support is preferred. Examples of alumina-based supports include but are not limited to, aluminum oxide, alumina, alumina monohydrate, alumina trihydrate, alumina-silica, bauxite, calcined aluminum hydroxides such as gibbsite, bayerite and boehmite, a-alumina, transition aluminas such as γ-alumina, η-alumina and δ-alumina, and calcined hydrotalcite.

The catalyst support has the following physical characteristics: an average particle size of 50 μm-5 mm, preferably 75 μm-1 mm, more preferably 100-500 μm; an average pore diameter of 1-100 nm, preferably 2-75 nm, more preferably 3-50 nm; a pore volume of 0.05-2.00 ml/g, preferably 0.10-1.50 ml/g, more preferably 0.20-1.00 ml/g; a surface area (which can be measured by Brunauer-Emmett-Teller of BET adsorption analysis) of 10-1000 m²/g, preferably 50-800 m²/g, more preferably 75-750 m²/g.

The catalyst support can be in the shape of wire gauzes, monoliths, particles, honeycombs, rings, etc. When the catalyst support is in the form of particles, the shape of the particles may include, but are not limited to, granules, beads, pills, pellets, cylinders, trilobes, spheres, irregular shapes, etc., as well as combinations comprising at least one of the foregoing

In addition to the dehydrogenation catalyst and the catalyst support, the catalytic composite according to the present invention includes a semimetal or a metalloid. Due to their unique properties that are intermediate those of metals and nonmetals, especially in electrical conductivity, semimetals have been applied extensively as semiconductors in modern electronic. Thermal or thermoelectric applications of semimetals, as they relate to catalyst functionality, have not been reported and metals have been the primary focus for such applications across different industries.

As used herein, a semimetal is chosen from boron, silicon, germanium, arsenic, antimony, tellurium, polonium, astatine and a combination thereof. Due to the toxicity or radioactivity of some of these semimetal elements, boron, silicon, germanium, antimony and tellurium are preferred.

As used herein, the semimetal acts as a heat-generating material with characteristics and properties as defined in U.S. Pat. No. 7,622,623. During the reduction stage of a Catofin cycle, wherein a catalyst bed is evacuated and reduced with hydrogen, the oxide of the semimetal (SMO) is reduced with the generation of heat (Equation 1). During the regeneration stage of the cycle which exposes the semimetal to an oxidizing condition, the reduced semimetal (SM) is converted to the oxide form providing an additional amount of heat (Equation 2):

SMO+H₂→SM+H₂O; ΔH<0 (Equation 1)

SM+O₂→SMO; ΔH<0 (Equation 2)

In other words, both the oxidation and reduction reactions of the semimetal are exothermic and accompanied by the release of heat.

The semimetal heat-generating material is inert towards dehydrogenation reactions including the hydrocarbon feed, the olefin products and other side reactions of the Catofin process such as cracking or coking. In this sense the term “inert” is used to mean that under the dehydrogenation conditions of the dehydrogenation reaction the semimetal does not catalyze the dehydrogenation of alkanes The semimetal, which is in direct contact with the dehydrogenation catalyst, does not participate in, is unaffected by, and/or is inactive, in the dehydrogenation reaction. For example, the semimetal is inert in a dehydrogenation reaction that produces propylene from propane. In the context of catalysts, where a metal/metal oxide catalyst useful in dehydrogenation reactions is mixed with the semimetal heat-generating material, the semimetal is considered to be inert and, as such, is understood to not directly affect, and not be directly affected by, the dehydrogenation reaction being catalyzed by the metal/metal oxide catalyst. However, without being bound by theory, it is believed that the semimetal may affect the conversion, selectivity, etc., of the oxidation reaction.

As used herein, the “alkane conversion” or simply “conversion” refers to the percentage of the total moles of feed (C₂-C₂₀, preferably, C₂-C₅ alkanes) that have been consumed by the reaction, i.e. the portion of the feed that has been consumed that is actually converted to the desired product (e.g. an olefin), regardless of other products. In general, selectivity is calculated as follows (Equation 3):

$$feed conversion ( \% ) = \frac{moles of feed converted}{moles of feed supplied} \times 100$$

The “selectivity of a particular product”, or simply “selectivity”, is the percentage of the percentage of the total moles of feed that have been consumed by the reaction, i.e. the portion of the feed that has been consumed that is actually converted to the desired product, regardless of other products. In general, selectivity is calculated as follows (Equation 4):

$$selectivity ( \% ) = \frac{moles of desired product produced}{moles of feed converted} \times \frac{\# of carbon atoms in product}{\# of carbon atoms in feed} \times 100$$

wherein # is number.

The “product yield”, or simply “yield”, as used herein, refers to the percentage of the total moles of the desired product (olefin) that would have been formed if all of the feed is converted to the product, as opposed to unwanted side products, e.g. acetic acid and COₓ compounds), and is generally calculated as follows (Equation 5):

$$product yield ( \% ) = \frac{moles of product produced}{moles of feed supplied} \times \frac{\# of carbon atoms in product}{\# of carbon atoms in feed} \times 100$$

The semimetal is reactive towards the reducing and/or oxidizing conditions of a Catofin process (reduction and regeneration stages). Further, the semimetal does not adversely affect the activity, selectivity or lifetime of the dehydrogenation catalyst. In certain embodiments, the inclusion of the heat-generating semimetal in the fixed catalyst bed can permit the use of lower air inlet temperatures to the reactor and reduce the combustion of coke and coke buildup, thereby eliminating the exposure of the olefin product to high temperatures that can otherwise result in byproduct formation and impairment of product selectivity. The generation of heat internal to a reactor reduces the necessity of additional heat supply through hot air and coke combustion, leading to the reduction of overall utility cost and increases the overall olefin yield for a given size of a reactor.

In certain embodiments, the semimetal is capable of generating, when combined from the dehydrogenation (oxidation) and regeneration (reduction) cycles, greater than (>) 700 kiloJoules per mole (kJ/mol) of heat in situ, preferably >750 kJ/mol, more preferably >800 kJ/mol, for example, 825-1,000 kJ/mol. In the following Table 1, which serves only as an example and is therefore not intended to limit the scope of the claims, the heat-generating capacity of a semimetal (antimony) is compared to the heat-generating capacities of different types of metals. In this example, the inventors have surprisingly discovered that the overall heat-generating capacity of antimony, as indicated by the net enthalpy in Table 1, exceeds those of magnesium, manganese, zinc, copper, bismuth and tin.

TABLE 1
Oxidation/reduction potentials and heat of reactions.

| | Oxidation | | | Reduction | | | |
|---|---|---|---|---|---|---|---|
| | | | Heat of | | | Heat of | |
| | | Potential, | reaction | | Potential, | reaction | Net |
| Element | Reaction | E⁰ (V) | (kJ/mol) | Reaction | E⁰ (V) | (kJ/mol) | enthalpy |
| H | 2H₂ + O₂ = 2H₂O | 0.00 | N/A | 2H₂O = 2H₂ + O₂ | 0.00 | N/A | N/A |
| Mg | Mg + ½O₂ = MgO | 2.37 | −601.8 | MgO = Mg + ½O₂ | −2.37 | +315.97 | −285.83 |
| Mn | Mn + ½O₂ = MnO | 1.18 | −770.7 | MnO = Mn + ½O₂ | −1.18 | +528.9 | −241.8 |
| | Mn + O₂ = MnO₂ | | −522.7 | MnO₂ = Mn + O₂ | | −49.3 | −572.0 |
| Zn | Zn + ½O₂ = ZnO | 0.74 | −350.46 | ZnO = Zn + ½O₂ | −0.74 | +64.63 | −285.83 |
| Cu | Cu + ½O₂ = CuO | −0.34 | −155.2 | CuO = Cu + ½O₂ | 0.34 | −130.64 | −285.84 |
| Bi | 2Bi + 3/2O₂ = | −0.215 | −573.9 | Bi₂O₃ + 3H₂ = | 0.215 | −283.59 | −857.49 |
| | Bi₂O₃ | | | 2Bi + 3H₂O | | | |
| Sn | Sn + ½O₂ = SnO | N/A | −286.2 | SnO = Sn + ½O₂ | N/A | +44.4 | −241.8 |
| | Sn + O₂ = SO₂ | | −580.7 | SnO₂ = Sn + O₂ | | +97.1 | −483.6 |
| Sb | 2Sb + 3/2O₂ = | −0.152 | −692.5 | Sb₂O₃ + 3H₂ = | 0.152 | −164.9 | −857.4 |
| | Sb₂O₃ | | | 2Sb + 3H₂O | | | |

Reduction of metal oxides by hydrogen depends on the standard reduction potential of the particular metals. Referring to Table 2 where standard reduction potentials at 25° C. of various elements and compounds are listed (including metals and nonmetals), the standard reduction potential of H₂ is considered being zero. Metals having positive standard reduction potential are considered as stronger oxidizing agents which means those metals can easily be reduced by hydrogen. However, metals having negative standard reduction potential are considered as stronger reducing agents which means those metals cannot be reduced by hydrogen. With such metals, reduction reactions are based on homolytic bond scission of the metal-oxide bond.

Table 3 compares the amount of heat released (corresponding with a temperature change expressed in ° C.) during oxidation and reduction cycles by antimony as a heat-generating semimetal and a commercial heat-generating metal. When the γ-Al₂O₃-supported antimony and the commercial heat-generating metal are individually combined with a commercial Cr-based standard catalyst composite, the former results in greater temperature increases in both the oxidation and reduction cycles.

TABLE 3
Comparative study of amount of heat released (° C.) during oxidation
& reduction cycle over antimony as a heat-generating semimetal with a commercial
heat-generating metal and composite materials (Space velocity = 400 h⁻¹)

| | | Temperature | Temperature | | |
|---|---|---|---|---|---|
| | Amount of | increase during | increase during | | |
| | material | Reduction by H₂ | Oxidation by | Heat of reaction (kJ/mol) | |
| Sample | taken (g) | (° C.) | Air (° C.) | Reduction | Oxidation |
| 12 wt. % Sb₂O₃/γ- | 5 | 7.8 | 14.6 | −164.9 | −692.5 |
| Al₂O₃ | | | | | |
| Commercial heat- | 5 | 8.5 | 8.4 | −130.6 | −155.2 |
| generating metal | | | | | |
| 12 wt. % Sb₂O₃/γ- | 2.5 + 2.5 | 10.4 | 18.5 | NA | NA |
| Al₂O₃ + commercial | | | | | |
| Cr-based standard | | | | | |
| catalyst composite | | | | | |
| Commercial heat- | 2.5 + 2.5 | 8.1 | 14.3 | NA | NA |
| generating metal + | | | | | |
| commercial ‘Cr-based | | | | | |
| standard catalyst | | | | | |
| composite | | | | | |

The average particle size of the semimetal (oxide or reduced form) is less than 1 μm, for example, 0.1-0.9 μm, preferably 0.25-0.75 μm, more preferably 0.35-0.65 μm. In certain embodiments, the semimetal particles are nanoparticles with an average particle size of less than 100 nm, preferably 20-80 nm, more preferably 35-75 nm.

To prepare the catalytic composite according to the present invention, any of the conventional methods of supported catalyst preparation such as wet impregnation and co-precipitation, may be used (Schwarz, J. A., “Methods for preparation of catalytic materials”, Chem. Rev. 1996(95): 477-510). In the wet impregnation method, a suspension of the solid catalyst support is treated with a solution of a dehydrogenation catalyst precursor such as a metal salt solution, followed by a solution of the semimetal precursor, and the resulting material is then activated to produce the catalytic composite. An example of a co-precipitation preparation process may involve treatment of an acidic solution of aluminum salts, dehydrogenation catalyst precursor and semimetal precursor with base to precipitate the mixed hydroxide, which is subsequently pelletized, dried, calcined and/or activated.

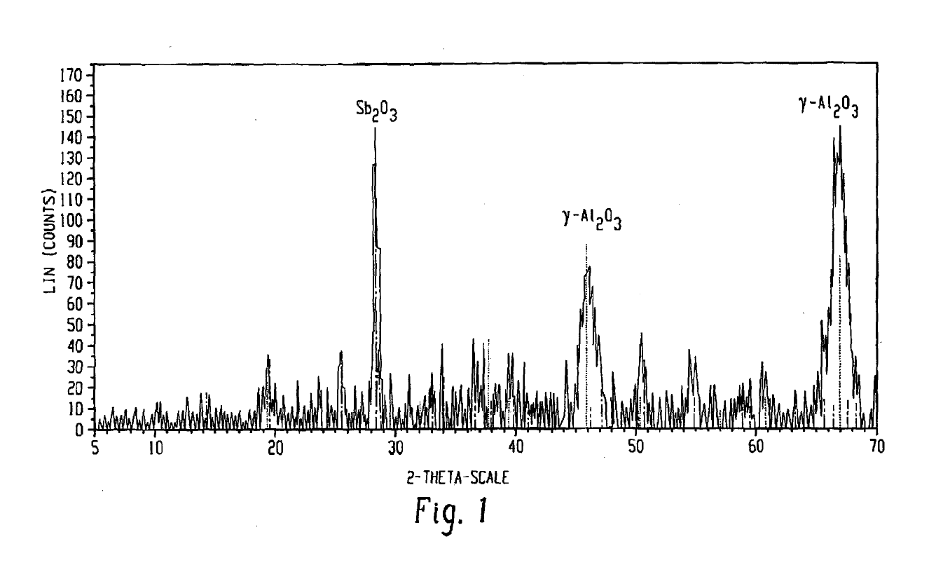

In one embodiment, antimony supported on γ-Al₂O₃ is prepared by depositing 10- 40 wt. % quantity of antimony acetate (antimony precursor) from an aqueous solution on the γ-Al₂O₃ catalyst carrier using an incipient wetness impregnating technique. Each catalyst support is treated with an aqueous solution of antimony precursor followed by drying and calcining in air at 700° C. for 4 h. The weight percentage of the antimony deposited is relative to the weight of the catalyst support. In FIG. 1, the XRD spectrum of calcined 12 wt. % Sb₂O₃/γ-Al₂O₃ showed formation of Sb₂O₃ phases after calcination.

Alternatively, the catalytic composite can be prepared by physically mixing or combining the dehydrogenation catalyst particles, the semimetal particles and loading the particle mixture onto the catalyst support without any chemical modification or thermal treatment such as drying and calcination.

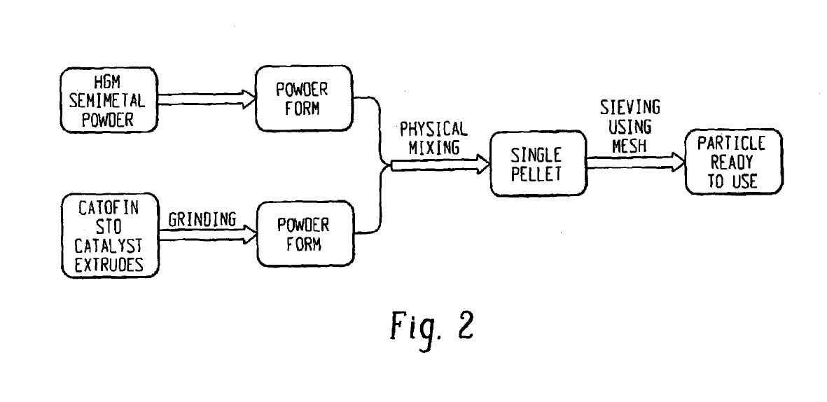

FIG. 2 illustrates a process of preparing a catalytic composite according to one embodiment. A commercial Catofin STD extruded catalyst is ground into powder form and then physically mixed with a heat-generating material semimetal powder. After that, the catalyst-semimetal powder mixture and the catalyst support are pelletized then sieved through a mesh.

In certain embodiments, the dehydrogenation catalyst constitutes 0.5-5.0 wt. % of the catalytic composite, preferably 1.0-4.0 wt. %, more preferably 1.0-3.0 wt. %. The semimetal constitutes 1-50 wt. % of the catalytic composite, preferably 2-40 wt. %, more preferably 5-35 wt. %. Alternatively, the semimetal constitutes 5-35 wt. % of the carrier, preferably 7- 30 wt. %, more preferably 8-25 wt. %. In one embodiment, the semimetal constitutes 24 wt. % of the carrier.

In some embodiments, the catalytic composite further comprises a promoter selected from lithium, sodium, potassium, rubidium, cesium, beryllium, magnesium, calcium, strontium, barium and a combination thereof. The effects of a promoter upon a dehydrogenation process can include, for example, enhanced alkane conversion, suppression of coke formation, elimination of high pre-heat temperature, improved stability of the dehydrogenation catalyst. A catalytic composite containing a promoter metal can be prepared by co-impregnation. A promoter may be made part of the catalyst composite, for example, being adsorbed to the surface of catalyst particles.

In some embodiments, the catalytic composite further comprises a binder that enhances the loading of the dehydrogenation catalyst, the semimetal and optionally the promoter onto the catalyst support. Binders may include silica such as colloidal silica, alumina, natural clay such as kaolin, kaolinite that may be modified, for example with at least one metal.

In some embodiments, the catalytic composite is substantially free of molybdenum, vanadium, yttrium, scandium, tungsten, manganese, cobalt, nickel, silver, bismuth, cerium, zinc, lead, indium, thallium, titanium, nickel, rhenium, selenium and lanthanum. As used herein, “substantially free” refers to a content of no more than 0.005 atomic percent (at. %) for each of the elements listed, preferably no more than 0.002 at. %, more preferably no more than 0.001 at. %.

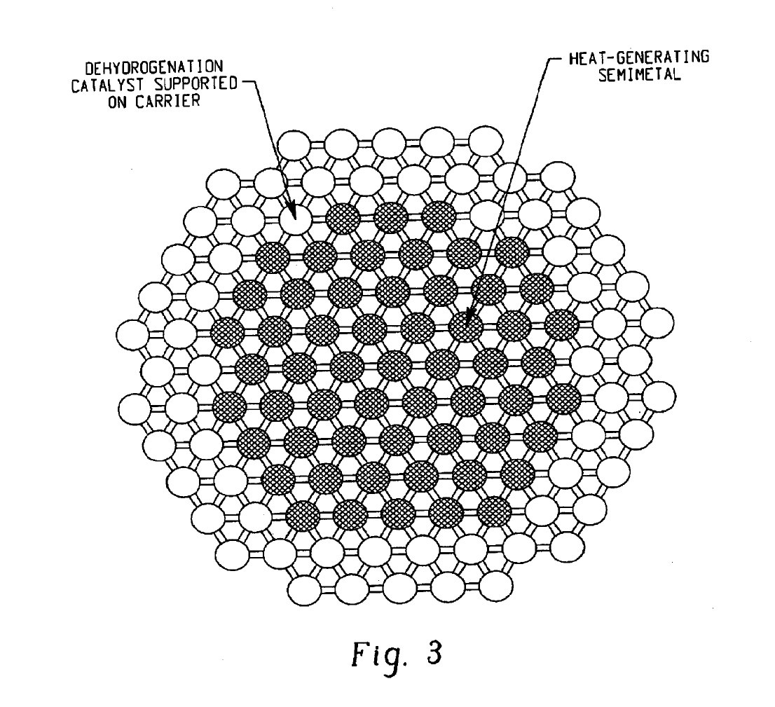

Further provided herein is a fixed catalyst bed system packed with at least one layer comprising the catalytic composite and a reactor comprising a fixed catalyst bed system packed with at least one layer comprising the catalytic composite. In one or more embodiments, the reactor is an adiabatic, fixed-bed reactor that is horizontally-oriented. FIG. 3 provides an example of the arrangement of the dehydrogenation catalyst, carrier and heat-generating semimetal in the catalyst bed, wherein the heat-generating semimetal material is disposed within the center of the bed and is surrounded by pelleted and supported dehydrogenation catalyst.

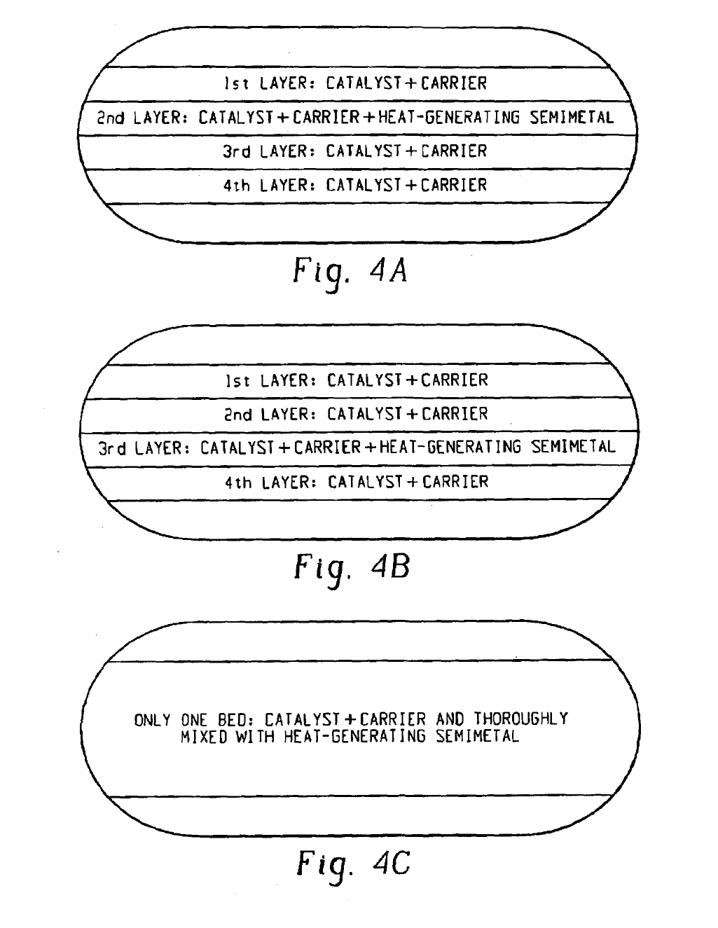

The fixed catalyst bed disclosed herein can include multiple layers (FIGS. 4A and 4B) where the heat-generating semimetal is present only in one or more of the inner or central layers. Alternatively, the heat-generating semimetal can be thoroughly mixed with a single layer of carrier-supported dehydrogenation catalyst particles (FIG. 4C).

An improved Catofin process is also provided. In the improved process, the catalyst bed comprising the catalytic according to the present invention is evacuated and reduced with hydrogen. During this stage, the semimetal releases heat that is passed evenly across the catalyst bed. An aliphatic hydrocarbon feed stream is then passed into and contacted with the catalyst bed to be dehydrogenated. The catalyst is then steam purged and regenerated (by oxidation) and the cycle is repeated with the evacuation/hydrogen reduction stage. During the regeneration stage, the semimetal can release additional heat that can be used to initiate the endothermic dehydrogenation of the following cycle.

In a typical Catofin process, the catalyst cokes up rapidly and therefore as many as five adiabatic, fixed-bed reactors are used in parallel. In the present disclosure, as coke combustion can be significantly reduced due to the presence semimetal heat-generating material in the catalyst bed, the number of reactors required for the Catofin process can also be reduced. In certain embodiments, the Catofin process according to the present disclosure operates with 2-4 adiabatic reactors.

In certain embodiments, the hydrocarbon feed stream may include an aliphatic hydrocarbon feed such as propane, n-butane, isobutylene and isopentane, air and a weak oxidant such as steam and/or carbon dioxide. In some embodiments, the hydrocarbon stream is substantially free of weak oxidants.

Set forth below are some embodiments of the catalyst composition, the process, and the reactors disclosed herein.

Embodiment 1: A catalytic composite suitable for a cyclic process of adiabatic, non-oxidative dehydrogenation of an alkane into an olefin, comprising: a dehydrogenation catalyst; a semimetal; and a carrier supporting the dehydrogenation catalyst and the semimetal; wherein the semimetal is inert towards the dehydrogenation, and releases heat in situ when exposed to at least one of a reducing stage and an oxidizing stage of the cyclic process.

Embodiment 2: The catalytic composite of Embodiment 1, wherein the semimetal is at least one of boron, silicon, germanium, arsenic, antimony, tellurium, polonium, and astatine.

Embodiment 3: The catalytic composite of any of the previous Embodiments, wherein the semimetal is a combination comprising at least one of boron, silicon, germanium, arsenic, antimony, tellurium, polonium, and astatine.

Embodiment 4: The catalytic composite of any of the previous Embodiments, wherein the semimetal is antimony.

Embodiment 5: The catalytic composite of any of the previous Embodiments 1, wherein the semimetal releases more than 700 kJ of heat per mole of the semimetal per reduction and oxidation cycle.

Embodiment 6: The catalytic composite of any of the previous Embodiments, wherein the semimetal is present in the catalytic composite in an amount of from 1 to 50 wt. % based on the total weight of the catalytic composite.

Embodiment 7: The catalytic composite of any of the previous Embodiments, wherein the semimetal has an average particle size of 0.25-0.75 μm.

Embodiment 8: The catalytic composite of any of the previous Embodiments, wherein the semimetal has an average particle size of 20-80 nm.

Embodiment 9: The catalytic composite of any of the previous Embodiments, further comprising a promoter supported on the carrier, the promoter is at least one of lithium, sodium, potassium, rubidium, cesium, beryllium, magnesium, calcium, strontium, and barium.

Embodiment 10: The catalytic composite of Embodiment 9, wherein the promoter is a combination comprising at least one of lithium, sodium, potassium, rubidium, cesium, beryllium, magnesium, calcium, strontium, and barium.

Embodiment 11: The catalytic composite of any of the previous Embodiments, further comprising a binder supported on the carrier.

Embodiment 12: The catalytic composite of any of the previous Embodiments, wherein the dehydrogenation catalyst is at least one of platinum that is optionally alloyed with tin; chromium, iron, and copper, oxides and mixtures and/or alloys thereof; and gallium, oxides and/or alloys thereof.

Embodiment 13: The catalytic composite of any of the previous Embodiments, wherein the dehydrogenation catalyst comprises at least one of platinum that is alloyed with tin.

Embodiment 14: The catalytic composite of any of the previous Embodiments, wherein the dehydrogenation catalyst comprises chromium, chromium oxide, a mixture comprising chromium, an alloy comprising chromium.

Embodiment 15: The catalytic composite of any of the previous Embodiments, wherein the dehydrogenation comprises iron, iron oxide, a mixture comprising iron, an alloy comprising iron.

Embodiment 16: The catalytic composite of any of the previous Embodiments, wherein the dehydrogenation comprises copper, copper oxide, a mixture comprising copper, an alloy comprising copper.

Embodiment 17: The catalytic composite of any of the previous Embodiments, wherein the dehydrogenation comprises gallium, gallium oxide, a mixture comprising gallium, and an alloy comprising gallium.

Embodiment 18: The catalytic composite of any of the previous Embodiments, wherein the dehydrogenation catalyst is chromium-based.

Embodiment 19: The catalytic composite of any of the previous Embodiments, wherein the carrier is at least one of alumina-based, magnesia-based, silica-based, zirconia-based, and zeolite-based.

Embodiment 20: The catalytic composite of any of the previous Embodiments, wherein the carrier is alumina-based, and preferably, the carrier is at least one of aluminum oxide, alumina, alumina monohydrate, alumina trihydrate, alumina-silica, bauxite, calcined gibbsite, calcined bayerite and calcined boehmite, α-alumina, γ-alumina, η-alumina and δ-alumina, and calcined hydrotalcite.

Embodiment 21: The catalytic composite of any of the previous Embodiments, wherein the catalytic composite is prepared by a method comprising at least one of wet impregnation, co-precipitation, and physical mixing.

Embodiment 22: The catalytic composite of any of the previous Embodiments wherein the catalytic composite is substantially free of cobalt, nickel, silver, bismuth, cerium, zinc, and lead.

Embodiment 23: The catalytic composite of any of the previous Embodiments wherein the catalytic composite is substantially free of indium, thallium, titanium, nickel, rhenium, selenium, and lanthanum.

Embodiment 24: The catalytic composite of any of the previous Embodiments wherein the catalytic composite is substantially free of molybdenum, vanadium, yttrium, scandium, tungsten, and manganese.

Embodiment 25: The catalytic composite of any of the previous Embodiments wherein the catalytic composite is substantially free of molybdenum, vanadium, yttrium, scandium, tungsten, manganese, cobalt, nickel, silver, bismuth, cerium, zinc, lead, indium, thallium, titanium, nickel, rhenium, selenium, and lanthanum.

Embodiment 26: A fixed bed catalyst packed with at least one layer comprising the catalytic composite of any of the previous Embodiments.

Embodiment 27: An adiabatic, fixed-bed reactor comprising a fixed bed catalyst packed with at least one layer comprising the catalytic composite of any of Embodiments 1 - 25.

Embodiment 28: A process of producing an olefin by adiabatic, non-oxidative dehydrogenation of an alkane, comprising:
(a) preparing a fixed bed catalyst comprising at least one layer of a catalytic composite, the catalytic composite comprising a dehydrogenation catalyst, a semimetal and a carrier supporting the dehydrogenation catalyst and the semimetal;
(b) reducing the fixed bed catalyst to generate a first heat supply, which is released by the semimetal, that is passed into the fixed catalyst bed;
(c) contacting a feed stream comprising the alkane with the fixed bed catalyst to endothermically dehydrogenate the alkane, wherein the thermal energy consumed by the dehydrogenation is at least partially provided by the first heat supply;
(d) steam purging and oxidizing the fixed bed catalyst to regenerate the fixed catalyst bed and oxidize the semimetal and to optionally generate a second heat supply; and
(e) optionally repeating (b) to (d) for multiple cycles.

Embodiment 29: The process of Embodiment 28, wherein the semimetal is antimony.

Embodiment 30: The process of any of Embodiments 28-29, wherein the semimetal releases more than 700 kJ of heat per mole of the semimetal per reduction/oxidation cycle.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combination” is inclusive of blends, mixtures, alloys, reaction products, and the like. Furthermore, the terms “first,” “second,” and the like, herein do not denote any order, quantity, or importance, but rather are used to denote one element from another; in other words, they are merely labels. Therefore, it is understood that the terms “first,” “second,” and the like, can be added as labels in the claims to prevent clarity issues. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the sheet(s) includes one or more sheets). Reference throughout the specification to “one embodiment,” “another embodiment,” “an embodiment,” and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments. Unless specified to the contrary herein, all test standards are the most recent standard in effect at the time of filing this application.

All cited patents, patent applications, articles, and other references are incorporated herein by reference in their entirety. However, if a term in the present application contradicts or conflicts with a term in the incorporated reference, the term from the present application takes precedence over the conflicting term from the incorporated reference. US Provisional Application No. 62/119,576, filed on Feb. 23, 2015, is hereby incorporated by reference in its entirety.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A catalytic composite suitable for a cyclic process of adiabatic, non-oxidative dehydrogenation of an alkane into an olefin, comprising:
a dehydrogenation catalyst;
a semimetal; and
a carrier supporting the dehydrogenation catalyst and the semimetal;
wherein the semimetal is inert towards the dehydrogenation, and releases heat in situ when exposed to at least one of a reducing stage and an oxidizing stage of the cyclic process.

2. The catalytic composite of claim 1, wherein the semimetal is at least one of boron, silicon, germanium, arsenic, antimony, tellurium, polonium, astatine and a combination thereof

3. The catalytic composite of claim 1, wherein the semimetal is antimony.

4. The catalytic composite of claim 1, wherein the semimetal releases more than 700 kJ of heat per mole of the semimetal per reduction and oxidation cycle.

5. The catalytic composite of claim 1, wherein the semimetal is present in the catalytic composite in an amount of from 1 to 50 wt. % based on the total weight of the catalytic composite.

6. The catalytic composite of claim 1, wherein the semimetal has an average particle size of 0.25-0.75 μm.

7. The catalytic composite of claim 1, wherein the semimetal has an average particle size of 20-80 nm.

8. The catalytic composite of claim 1, claims, further comprising a promoter supported on the carrier, the promoter is at least one of lithium, sodium, potassium, rubidium, cesium, beryllium, magnesium, calcium, strontium, and barium.

9. The catalytic composite of claim 1, further comprising a binder supported on the carrier.

10. The catalytic composite of claim 1, wherein the dehydrogenation catalyst is at least one of platinum that is optionally alloyed with tin; chromium, iron, and copper, oxides and mixtures and/or alloys thereof; and gallium, oxides and/or alloys thereof

11. The catalytic composite of claim 1, wherein the dehydrogenation catalyst is chromium-based.

12. The catalytic composite of claim 1, wherein the carrier is at least one of alumina-based, magnesia-based, silica-based, zirconia-based, and zeolite-based.

13. The catalytic composite of claim 1, wherein the carrier is alumina-based and is at least one of aluminum oxide, alumina, alumina monohydrate, alumina trihydrate, alumina-silica, bauxite, calcined gibbsite, calcined bayerite and calcined boehmite, α-alumina, γ-alumina, η-alumina and δ-alumina, and calcined hydrotalcite.

14. The catalytic composite of claim 1, wherein the catalytic composite is prepared by a method comprising at least one of wet impregnation, co-precipitation, and physical mixing.

15. The catalytic composite of claim 1 wherein the catalytic composite is substantially free of molybdenum, vanadium, yttrium, scandium, tungsten, manganese, cobalt, nickel, silver, bismuth, cerium, zinc, lead, indium, thallium, titanium, nickel, rhenium, selenium, and lanthanum.

16. A fixed bed catalyst packed with at least one layer comprising the catalytic composite of any of claim 1.

17. An adiabatic, fixed-bed reactor comprising a fixed bed catalyst packed with at least one layer comprising the catalytic composite of claim 1.

18. A process of producing an olefin by adiabatic, non-oxidative dehydrogenation of an alkane, comprising:
(a) preparing a fixed bed catalyst comprising at least one layer of a catalytic composite, the catalytic composite comprising a dehydrogenation catalyst, a semimetal and a carrier;
(b) reducing the fixed bed catalyst to generate a first heat supply, which is released by the semimetal, that is passed into the fixed bed catalyst;
(c) contacting a feed stream comprising the alkane with the reduced fixed bed catalyst to endothermically dehydrogenate the alkane, wherein the thermal energy consumed by the dehydrogenation is at least partially provided by the first heat supply;
(d) steam purging and oxidizing the fixed bed catalyst to regenerate the fixed bed catalyst and oxidize the semimetal and to optionally generate a second heat supply; and
(e) optionally repeating (b) to (d) for multiple cycles.

19. The process of claim 18, wherein the semimetal is antimony.

20. The process of claim 18, wherein the semimetal releases more than 700 kJ of heat per mole of the semimetal per reduction/oxidation cycle.
